(19) United States
(12) Patent Application Publication
(10) Pub. No.: US 2016/0095584 A1
(43) Pub. Date: Apr. 7, 2016

(54) ENDOSCOPIC NEEDLE WITH ROTARY JAW FOR LATERAL ACQUISITION

(71) Applicant: Boston Scientific Scimed, Inc., Maple Grove, MN (US)

(72) Inventors: Brian Almeida, Newton, MA (US); Paul Smith, Smithfield, RI (US)

(21) Appl. No.: 14/860,111

(22) Filed: Sep. 21, 2015

Related U.S. Application Data
(60) Provisional application No. 62/058,335, filed on Oct. 1, 2014.

(51) Int. Cl. A61B 10/06; A61B 1/018; A61B 10/02

(52) U.S. Cl. CPC A61B 10/06; A61B 10/0275; A61B 1/018; A61B 2010/045

(57) ABSTRACT

A device for collecting a tissue sample includes a needle extending along a longitudinal axis from a proximal end to a distal end and including a lumen extending therethrough; an opening in a lateral surface of the needle; and a jaw rotatably coupled to the needle for rotation between a closed configuration in which the jaw covers the opening and an open configuration in which the jaw is received within the lumen to expose the opening. The jaw includes a tissue cutting edge which, as the jaw is rotated from the open configuration to the closed configuration, passes out of the opening along a cutting arc to sever any tissue received in the opening from surrounding tissue.

### PRIORITY CLAIM

The present application claims priority to U.S. Provisional Patent Application Ser. No. 62/058,335 filed Oct. 1, 2014.

### BACKGROUND

Needle biopsy procedures may be used for the diagnosis and the staging of disease. For example, a fine needle aspiration needle may be advanced through a working channel of an endoscope into the body to the location of target tissue to be sampled. Although fine needle aspiration is a highly sensitive and specific procedure, it may be difficult to acquire a suitable sample in certain clinical situations. The more cells or tissue that can be acquired, the greater the potential for a definitive diagnosis. Although larger gauge needles may be used to obtain larger samples, they may be difficult to pass along tortuous paths to target sites and may acquire samples including more blood, making it more difficult to obtain an accurate diagnosis.

### SUMMARY

The present disclosure is directed to a device for collecting a tissue sample, comprising a needle extending along a longitudinal axis from a proximal end to a distal end and including a lumen extending therethrough and an opening in a lateral surface of the needle in combination with a jaw rotatably coupled to the needle for rotation between a closed configuration in which the jaw covers the opening and an open configuration in which the jaw is received within the lumen to expose the opening, the jaw including a tissue cutting edge which, as the jaw is rotated from the open configuration to the closed configuration, passes out of the opening along a cutting arc to sever any tissue received in the opening from surrounding tissue.

In an embodiment, the jaw may include an outer, convex surface which, in the closed configuration, faces outward from the needle to form a portion of an outer surface of the needle and an inner, concave surface which forms a scoop for gathering tissue severed by the cutting edge and trapping the severed tissue within the lumen.

In an embodiment, the jaw may be sized and shaped so that, when rotating between open and closed configurations, the cutting edge projects laterally outward from the opening in the needle.

In an embodiment, the jaw may be coupled to an actuation mechanism extending through the device to a proximal end of the needle which, during use, remains accessible to a user.

In an embodiment, the jaw may be coupled to the needle via a hinge.

In an embodiment, the actuation mechanism may be a flexible longitudinal element coupled to an end of the jaw which, when the jaw is in the open configuration, is located distally of the hinge.

In an embodiment, the flexible longitudinal element may be wound about the jaw so that the jaw may be moved from the open configuration to the closed configuration and subsequently moved from the closed configuration to the open configuration.

In an embodiment, the opening may be adjacent to a distal end of the needle.

In an embodiment, the jaw may be shaped as a portion of an ellipsoid.

In an embodiment, the jaw may be shaped as a portion of an sphere.

In an embodiment, the jaw may include a central portion shaped as a portion of an ellipsoid with planar lateral sides extending substantially parallel to a longitudinal axis of the needle.

The present disclosure is also directed to a system for collecting a tissue sample, comprising a needle extending along a longitudinal axis from a proximal end to a distal end and including a lumen extending therethrough and an opening in a lateral surface of the needle in combination with a jaw rotatably coupled to the needle for rotation between a closed configuration in which the jaw covers the opening and an open configuration in which the jaw is received within the lumen to expose the opening, the jaw including a tissue cutting edge which, as the jaw is rotated from the open configuration to the closed configuration, passes out of the opening along a cutting arc to sever any tissue received in the opening from surrounding tissue, and an endoscope with a working channel through which the needle is received.

In an embodiment, the jaw may be coupled to the needle via a hinge.

In an embodiment, the jaw may include an outer, convex surface which, in the closed configuration, faces outward from the needle to form a portion of an outer surface of the needle and an inner, concave surface which forms a scoop for gathering tissue severed by the cutting edge and trapping the severed tissue within the lumen.

The present disclosure is also directed to a method for collecting a tissue sample, comprising inserting a needle to a target tissue within a living body, the needle extending along a longitudinal axis from a proximal end to a distal end and including a lumen extending therethrough, drawing tissue into the lumen through an opening in a lateral surface of the needle, and rotating a jaw rotatably coupled to the needle so that a tissue cutting edge of the jaw passes out of the opening along a cutting arc to sever the tissue received in the opening from surrounding tissue.

In an embodiment, the jaw may be coupled to the needle via a hinge and wherein the jaw includes a concave inner surface which faces the hinge.

In an embodiment, the jaw may include an opening therein through which sampled tissue may pass into the needle lumen.

In an embodiment, during insertion of the device through the body to a target site, the jaw is maintained in the closed configuration and, when the target site is reached, the jaw may be rotated from the closed configuration to the open configuration to expose the opening.

In an embodiment, suction may be applied to the lumen of the needle to draw tissue into the opening.

In an embodiment, the jaw may be rotated by pulling on a flexible longitudinal element extending from a proximal end of the needle to couple to the jaw.

### BRIEF DESCRIPTION OF THE DRAWINGS

FIG. 1 shows a top view of a distal portion of a device according to an exemplary embodiment of the present disclosure;

FIG. 2 shows an isometric view of the device of FIG. 1.

FIG. 3 shows a longitudinal view of the device of FIG. 1;

FIG. 4 shows a longitudinal cross-sectional view of the device of FIG. 1;

FIG. 5 shows a perspective view of the device of FIG. 1 from the distal end of the device;

FIG. 6 shows another perspective view of the device of FIG. 1 from the proximal end of the device;

### DETAILED DESCRIPTION

The present disclosure may be further understood with reference to the following description and the appended drawings, wherein like elements are referred to with the same reference numerals. The present disclosure is related to endoscopic devices and, in particular, devices for obtaining tissue samples. Exemplary embodiments of the present disclosure describe a needle comprising

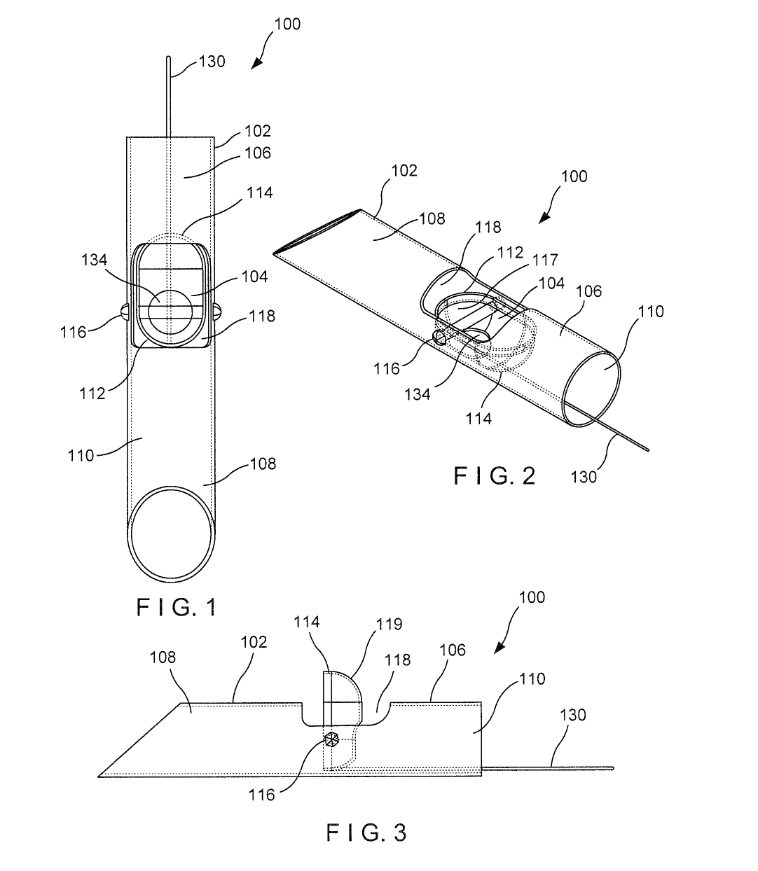

As shown in FIGS. 1-6, a system 100 for collecting a tissue sample according to an exemplary embodiment of the present disclosure comprises a needle 102 including a rotary jaw 104 pivotally coupled to a distal portion 108 of a needle body 106 via a hinge 116. The rotary jaw 104 rotates in an opening 118 formed in a distal portion of the needle 102. The jaw 104 is coupled to an actuation mechanism 130, for example, a pull-wire, that rotates the jaw 104 about the hinge 116 between an open and a closed position. The jaw 104, according to an embodiment of the disclosure is a curved shape that forms a scoop within which a tissue sample may be gathered. For example, in this embodiment, the jaw 104 may be a portion of an ellipsoid (e.g., half an ellipsoid). Those, skilled in the art will understand that many other shapes may be employed for the jaw 104 such as a portion of a sphere or a scoop with planar walls parallel to a longitudinal axis of the needle 102 on either side of a curved central portion. Thus, the jaw 104 extends along a longitudinal curve in a proximal to distal direction and is also curved in planes perpendicular to the longitudinal curve so that a radially inner surface thereof is concave. The jaw 104 is rotatable about the hinge 116 between an open configuration in which the jaw 104 is positioned within the lumen 110 with the concave inner surface 117 of the jaw 104 facing out of the opening 118 and a closed position in which the jaw 104 covers the opening 118 with the convex outer surface 119 thereof facing outward. As will be described in more detail below, as the jaw 104 is rotated from the open position to the closed position by the actuation mechanism 130, a cutting edge 114 at a first end of the jaw 104 passes along a cutting arc to sever tissue that has been drawn into lateral opening 118 (e.g., via suction). Continued rotation of the jaw 104 traps the severed tissue within the lumen 110 from which it may be withdrawn via suction or other means as would be understood by those skilled in the art. As will be described below in more detail, the jaw 104 is maintained in the closed configuration as the needle 102 is inserted into the body and, when the needle 102 has reached a target position adjacent to tissue to be sampled, the jaw 104 is rotated to the open configuration to open the lateral opening 118. Target tissue is then drawn into the opening 118 by, for example, apply suction through the lumen 110. The jaw 104 is then rotated to the closed configuration drawing the cutting edge 114 through the tissue to sever a sample which, as the jaw 104 reaches the closed configuration, is trapped in the lumen 110. As shown specifically in FIGS. 5 and 6 and described in more detail below, the system 100 may further comprise a source of vacuum pressure for drawing the tissue sample through the opening 118 into the needle 102 and for aspirating a sample through the lumen to the proximal end of the needle 102 where it may be collected. The process may then be repeated to gather additional tissue samples.

The needle body 106 extends longitudinally from a proximal end (not shown) to the distal end 108 and includes the lumen 110 extending therethrough along the longitudinal axis thereof. The needle body 106 may be sized and shaped to be received through a working channel of an endoscope or other insertion device and is preferably sufficiently flexible to pass through the endoscope as it is inserted through a tortuous path into a living body, for example, under ultrasound guidance. The distal end 108 may be tapered, beveled or otherwise shaped as desired to facilitate insertion to the target tissue area. Alternatively, the distal end may be rounded to prevent damage to non-targeted tissue adjacent to the tissue to be sampled.

The jaw 104 extends from a first end 112 to a cutting end 114. The jaw 104 in this embodiment is shaped as a hemi-ellipsoid including a recess or chamber so that when the jaw 104 rotates about the hinge 116 the first end 112 of the jaw 104 cuts the target tissue 10 to collect it within the recess. In other embodiments, the jaw 104 may be hemi-spherical or hemi-ellipsoidal with flat lateral sides. The jaw 104 in this embodiment is sized and shaped to fit the contours of the needle body 106. The jaw 104 is rounded along the longitudinal axis from the first end 112 to the cutting edge 114 and along an axis perpendicular to the longitudinal axis to form a recess therein. In particular, the jaw, 104 may be sized and shaped so that, when it is rotated to the closed configuration, the jaw 104 covers the lateral opening of the lumen. The jaw 104 is laterally connected to the needle body 106 via a hinge 116 about which the jaw is fully rotatable. Although exemplary embodiments show and describe the hinge as fully rotatable, it will be understood by those of skill in the art that the hinge 116 may be formed as any of a variety of types of hinge joints so long as the jaw 104 is able to pivot thereabout between the closed and open configuration. As described above, the jaw 104 may be positioned at the distal end 108 of the needle body 106 or immediately proximally thereto. The jaw 104 may optionally have an opening 134 extending therethrough to allow sampled tissue to be suctioned by the vacuum source from the jaw 104 into the lumen 110 of the needle 102. The opening 134 is positioned so that, when in the open configuration, the opening 134 is aligned with the longitudinal axis.

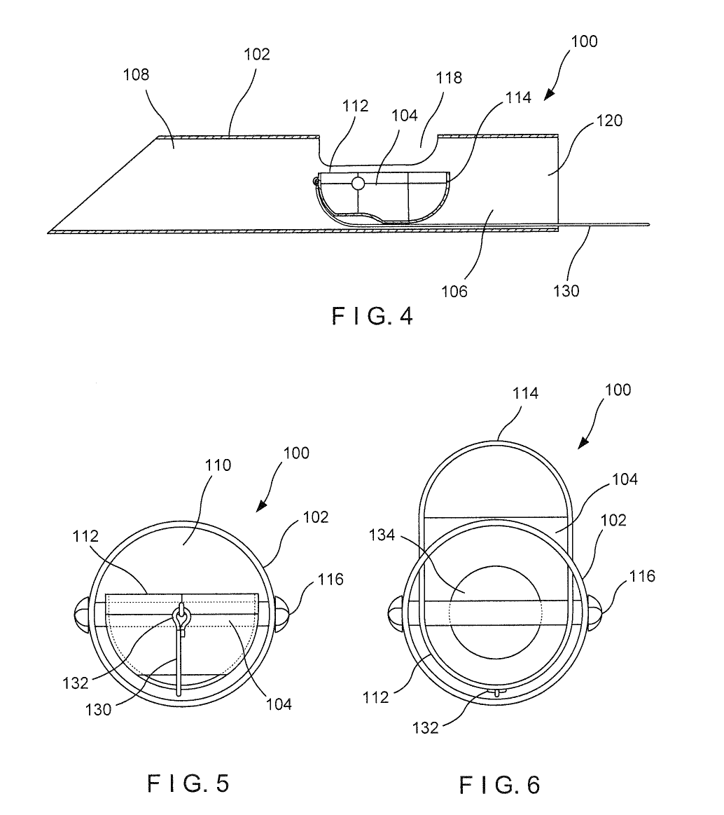

In an exemplary embodiment see in FIGS. 4-5, the jaw 104 is rotated via an actuation mechanism 130, for example a pull-wire coupled to the first end 112 of the jaw 104 via a coupling element 132. Although exemplary embodiments show and describe the actuation mechanism as a pull-wire, it will be understood by those of skill in the art that various other actuation mechanisms may be used and that the pull-wire actuator need not be a wire but may be any flexible longitudinal element sufficiently strong so as to enable it to rotate the jaw 104 through the target tissue. According to this embodiment, when the needle 102 is positioned adjacent to or within target tissue 10, the actuation mechanism is operated to rotate the jaw 104 from the closed configuration to the open configuration and suction is applied to draw tissue into the opening 118. The actuation mechanism is then further operated to rotate the jaw 104 from the open configuration to the closed configuration severing the tissue drawn into the opening 118 from the surrounding tissue and trapping the severed tissue within the lumen 110. Suction may then be used to aspirate this tissue through the lumen 110 to the proximal end of the needle where it maybe withdrawn for analysis. Alternatively, the needle 102 may be withdrawn from the body and the tissue may be manually removed from the needle 102 via the opening 118.

Variations may be made in the structure and methodology of the present disclosure, without departing from the spirit and the scope of the disclosure. Thus, it is intended that the present disclosure cover the modifications and variations of this disclosure that may be contemplated by a person skilled in the art.

1-15. (canceled)

16. A device for collecting a tissue sample, comprising:
a needle extending along a longitudinal axis from a proximal end to a distal end and including a lumen extending therethrough;
an opening in a lateral surface of the needle; and
a jaw rotatably coupled to the needle for rotation between a closed configuration in which the jaw covers the opening and an open configuration in which the jaw is received within the lumen to expose the opening, the jaw including a tissue cutting edge which, as the jaw is rotated from the open configuration to the closed configuration, passes out of the opening along a cutting arc to sever any tissue received in the opening from surrounding tissue.

17. The device of claim 16, wherein the jaw includes an outer, convex surface which, in the closed configuration, faces outward from the needle to form a portion of an outer surface of the needle and an inner, concave surface which forms a scoop for gathering tissue severed by the cutting edge and trapping the severed tissue within the lumen.

18. The device of claim 16, wherein the jaw is sized and shaped so that, when rotating between open and closed configurations, the cutting edge projects laterally outward from the opening in the needle.

19. The device of claim 16, wherein the jaw is coupled to an actuation mechanism extending through the device to a proximal end of the needle which, during use, remains accessible to a user.

20. The device of claim 19, wherein the jaw is coupled to the needle via a hinge.

21. The device of claim 20, wherein the actuation mechanism is a flexible longitudinal element coupled to an end of the jaw which, when the jaw is in the open configuration, is located distally of the hinge.

22. The device of claim 21, wherein the flexible longitudinal element is wound about the jaw so that the jaw may be moved from the open configuration to the closed configuration and subsequently moved from the closed configuration to the open configuration.

23. The device of claim 16, wherein the opening is adjacent to a distal end of the needle.

24. The device of claim 16, wherein the jaw is shaped as a portion of an ellipsoid.

25. The device of claim 16, wherein the jaw is shaped as a portion of a sphere.

26. The device of claim 16, wherein the jaw includes a central portion shaped as a portion of an ellipsoid with planar lateral sides extending substantially parallel to a longitudinal axis of the needle.

27. The device of claim 16, wherein the jaw includes a central portion shaped as a portion of a sphere with planar lateral sides extending substantially parallel to a longitudinal axis of the needle.

28. A system for collecting a tissue sample, comprising:
a needle extending along a longitudinal axis from a proximal end to a distal end and including a lumen extending therethrough;
an opening in a lateral surface of the needle;
a jaw rotatably coupled to the needle for rotation between a closed configuration in which the jaw covers the opening and an open configuration in which the jaw is received within the lumen to expose the opening, the jaw including a tissue cutting edge which, as the jaw is rotated from the open configuration to the closed configuration, passes out of the opening along a cutting arc to sever any tissue received in the opening from surrounding tissue; and
an endoscope with a working channel through which the needle is received.

29. The system of claim 28, wherein the jaw includes an outer, convex surface which, in the closed configuration, faces outward from the needle to form a portion of an outer surface of the needle and an inner, concave surface which forms a scoop for gathering tissue severed by the cutting edge and trapping the severed tissue within the lumen.

30. A method for collecting a tissue sample, comprising:
inserting a needle to a target tissue within a living body, the needle extending along a longitudinal axis from a proximal end to a distal end and including a lumen extending therethrough;
drawing tissue into the lumen through an opening in a lateral surface of the needle; and
rotating a jaw rotatably coupled to the needle so that a tissue cutting edge of the jaw passes out of the opening along a cutting arc to sever the tissue received in the opening from surrounding tissue.

31. The method of claim 30, wherein the jaw is coupled to the needle via a hinge and wherein the jaw includes a concave inner surface which faces the hinge.

32. The method of claim 30, wherein the jaw includes an opening therein through which sampled tissue may pass into the needle lumen.

33. The method of claim 30, further comprising, during insertion of the device through the body to a target site, maintaining the jaw in the closed configuration and, when the target site is reached, rotating the jaw from the closed configuration to the open configuration to expose the opening.

34. The method of claim 30, wherein suction is applied to the lumen of the needle to draw tissue into the opening.

35. The method of claim 30, wherein the jaw is rotated by pulling on a flexible longitudinal element extending from a proximal end of the needle to couple to the jaw.
